(19) United States
(12) Patent Application Publication
(10) Pub. No.: US 2003/0108877 A1
(43) Pub. Date: Jun. 12, 2003

(54) Methods for selecting and producing selective pharmaceutical compounds and compositions using an established genetically altered cell-based library responsive to transcription factors; genetic constructs and library therefor.

(76) Inventors: Yves Blais, Montreal (CA); Pierre Rousseau, Longueuil (CA); Patricia Villain, Montreal (CA); Benoit LeBlanc, Montreal (CA); Rino N Camato, St-Leonard (CA)

Correspondence Address: MERCHANT & GOULD PC, P.O. BOX 2903, MINNEAPOLIS, MN, 55402-0903, US

(21) Appl. No.: 10/030,929

(22) PCT Filed: Dec. 21, 2001
(86) PCT No.: PCT/CA01/01861

(30) Foreign Application Priority Data
Dec. 27, 2000 (CA) 2327581

(51) Int. Cl. C12Q 1/70; C12Q 1/68; G01N 33/53; G01N 33/567; C12N 15/867

(52) U.S. Cl. 435/6; 435/5; 435/7.2; 435/456

(57) ABSTRACT

The methods and products that are described in the present patent application are designed to determine by serial analysis the transcriptional activation pathways used by any compound of interest that is biologically active in a cell. We have engineered a library of transgenic or recombinant cell lines that allow the identification of simultaneous transcriptional pathways possibly used by the compound. Each cell line constituting the library has been engineered by stable transfection thereof by retroviral integration, to carry a reporter gene under the transcriptional control of a particular transcription factor or family thereof endogenous to the host cell. This is achieved by having the expression of the reporter gene driven by a minimal promoter and a transcription factor responsive-element (TF-RE) or a tandem or concatemer thereof. In its complete form, the library contains a large collection of reporter constructs responding to individual factors, each of which can in turn be studied in a large repertory of cell lines. Such an approach provides a global view of gene transcription activation in response to diverse stimuli in multiple cellular environments and is a significant improvement over case-by-case pproaches. A compound screened against such a library provides a profile of selectivity for a given transcription factor and for a given cell type, which profile is used to select good selective putative therapeutics.

### FIELD OF THE INVENTION

This invention relates to a method for selecting and producing selective pharmaceutical compounds, which include monitoring the activity of compounds on transcriptional activity in a cell library expressing a construct comprising a transcription-factor responsive promoter element and a reporter gene.

### BACKGROUND OF THE INVENTION

The human genome is composed of roughly 30 to 40 000 genes, with roughly 5% of these are believed to encode regulators that include transcription factors and their associated proteins and cofactors (International Human Genome Sequencing Consortium, 2001). The control of gene expression is mostly regulated at the transcriptional level by these regulators. Most of these regulatory factors are expressed in an histospecific manner meaning that, for a given cell in the organism, a specific group of regulatory proteins will be expressed to confer to that cell the pattern of gene expression appropriate to its nature and function. The level of expression of such regulatory proteins as well as their activity is also tightly regulated depending on cell identity and cell state. When this typical pattern is disturbed, the ensuing deregulation of gene expression may result in altered cell behaviour or phenotype and in a pathological state. The regulators, transcription factors in particular, bind to DNA on short and defined sequences in a specific manner according to a lock-and-key principle defined by the factor's architecture and electrostatic interactions between the transcription factor and DNA. Once tethered to DNA, transcription factors activate, stabilize (or does not alter), and/or repress gene expression. Because of the specificity of transcription factors for their DNA binding site and because of the increasing knowledge of the consensus sequences of these sites, it is possible to predict where on a fragment of DNA a given transcription factor should bind.

The transcription factors are at the forefront of gene regulation. They control genetic switches that lead to simultaneous expression of genes in response to different stimuli. Drugs can have an effect on transcription factors in many ways: they can bind to membrane receptors at the surface of the cell and trigger signalling cascades which will ultimately induce a chemical modification of a transcription factor molecule or one of its cofactors; they can enter the cell and directly contact a transcription factor molecule to trigger an effect such as a conformational change. Such a change can play many roles in the behaviour of the transcription factor: it can modify the affinity of the factor for DNA; it can change its affinity for co-activator or co-repressor partners; it can modify its activation domain so as to make it more active or less active as an activator of transcription. The resulting change in gene expression triggered by the effect of the drug on a particular factor can have a major impact on cell viability, differentiation, response and apoptosis. Many studies have focused on the role certain drugs can play as inducers or repressors of transcription factors, and on the therapeutic consequences which can be derived therefrom (Smith and Birrer, 1996; Lehmann et al., 1997; Henke et al., 1998; Brown et al., 1999;; Kliewer et al., 2001; Oliver et al., 2001).

Numberous transcription factors have therefore been proposed as therapeutic agents or targets. Among many possible examples, let us mention a few ones. The transfer of gene coding for the wild-type form of the p53 transcription factor using a retroviral vector in non-small cell lung cancer patients has been shown to induce tumor regression and tumor growth stabilization (Roth et al., 1996). The administration of NFkB antisense oligonucleotide in mice (NFkB being another transcription factor) has eliminated induced colitis (Neurath et al., 1996). The NFAT protein, another transcription factor, is known to regulate the expression of many immune response modulators such as interleukins and interferons (Rao et al., 1997; Chow et al., 1999). NFAT is found in its inactive and phosphorylated form in the cytoplasm until the unmasking of its Nuclear Localization Signal by the action of the phosphatase calcineurin allows it to migrate to the nucleus and bind DNA. It is because the cyclosporin molecule inhibits calcineurin that it can be used as an immunosupressant drug: it interferes with the action of NFAT (Clipstone et al., 1992; Jain et al., 1993; Kubo et al., 1994; Nair et al., 1994). Steroidogenic factor-1 (SF-1), another transcription factor, activates the aromatase p450 promoter by displacing the COUP-TF transcription factor and, consequently, causes the conversion of estrone into estradiol, which in turn activates a set of genes involved in endometriosis (Zeitoun et al., 1999).

Practical Considerations.

The transcriptional adaptation of cells to the action of a compound can be monitored in many ways, either directly or indirectly. The use of fluorescent or luminescent reporter genes to monitor gene regulation as been described in both academic and industrial literature (Riggs and Chrispeels, 1987; Waterman et al., 1988; Nordeen, 1988; DiLella et al., 1988; Baulcombe et al., 1995; Barthmaier and Fryberg, 1995; Marshall et al., 1995; Yeh et al., 1995). Even more relevant to the current patent application is the report of a stably transfected Jurkat cell line expressing as a reporter marker a recombinant protein, the green fluorescent protein or GFP, whose gene is under the control of the NFAT transcription factor (Hooijberg et al., 2000). This publication does not teach however how to build a library of different cell types transformed with a plurality of transcription factors for the purpose of evaluating or predicting which screened compound is selective for a factor and/or a cell type, and which is a candidate as a selective therapeutic.

### SUMMARY OF THE INVENTION

The basis of the current patent application is the development and use of a system in which the activity of a compound is tested not merely on one transcription factor and/or in one type of cell line, but on a multiplicity of factors and in multiple different cell lines in a parallel study. Such a system called hereinbelow “Cell-TRAP” allows the study of the effects of a potential drug, for example, on the activity of as many different transcription factors associated with a given pathology, and in as much cell types also associated with said pathology as possible (if not all). This very high-throughput system allows a global view of the effects of a compound on transcription and is a distinct improvement over individual systems which would be limited to certain aspects of gene activation. Furthermore, the system can be used as a predictive tool to evaluate the possibility of occurrence of side-effects of a compound (which in this case would be the activation of undesirable transcription activation pathways in certain cell types). The ultimate goal is to find a drug candidate which has no serious side effect, thus not affecting non-targeted TFs or TFREs. Since the transcriptional pathways studied with the present method and products mimick a natural pathway, chances are greater to identify a compound which would be selective in vivo, thus a valuable therapeutic.

The present invention relates to a construct to transform cells of different types, a library of recombinant cells comprising the construct, methods of making thereof and methods of use thereof, the library expressing a DNA construct comprising a known transcription factor responsive-element (TFRE) operably linked to a minimal promoter and to a reporter gene. The expression of the reporter gene governed by the promoter and the TFRE which is activated, non-activated or repressed upon binding by a transcription factor. Upon screening the library with a compound of interest, a cascade of events triggers the synthesis of a transcription factor and its binding to a TFRE. A difference in the expression of the reporter gene indicates that this candidate compound has an effect on a known TFRE, and on an assorted TF. The screening is conducted on different cell lines and on cell lines of different species such as human, rat, mouse, insect, plant, and monkey.

The screening allows screening of a wide range of compounds either natural or synthetic and it allows the investigation of transcriptional regulatory activity of a compound of interest, whereas the compound is lipid, protein, deoxyrinonucleic acid, ribonucleic acid, polycyclic carbones, steroid, or else, and this, simultaneously on a diversity of cell lines.

The activity of the reporter gene is monitored by methods using a fluorescent, or a luminescent reporter gene, or is monitored by methods using immunological detection of an antigen, or is monitored by methods using polymerase chain reaction specific DNA primers. The primers could be directed against the reporter gene sequence itself or the flanking sequences that would be co-expressed with the reporter gene. The primers themselves would be labelled or a probe directed against the amplified sequence could provide the label or a detection means member.

The screening allows the determination of the level of pathway specificity of a given compound used as a potential activator or repressor of transcriptional activity. The specificity of action of the compound on a given transcription factor is evaluated by comparing the activity of many different transcription factors in genetically modified stable cell lines of similar tissue origin.

Additionally or alternatively, the screening allows the determination of how a given substance affects a particular transcriptional activation or repression pathway in tissues of different origins.

Therefore, in accordance with the present invention is provided a method for selecting and producing a therapeutic compound which is presumed selective for one or a restricted set of given transcriptional pathways and of given cell types, which comprises:

providing a construct which comprises a reporter gene, the expression of which is driven in a host cell by a promoter capable of directing transcription of the gene operably linked thereto upon activation, which promoter comprises a minimal promoter and, upstream to said minimal promoter, a transcription factor-responsive element which is capable of affecting the activity of the minimal promoter upon binding by a transcription factor endogenously produced, activated or inactivated by the host cell upon contacting by the compound; the construct being provided for a plurality of transcription-factor responsive elements for a given cell line and for a plurality of cell lines representative of different tissues;

inserting each construct into the genome of the host cell of each cell line, thereby obtaining a library of recombinant cell lines;

contacting the compound with the library of recombinant cell lines;

detecting a change in the expression of the reporter gene occurring in one recombinant cell line or in a subset of recombinant cell lines and not in other cell lines of the library as an indication of a putative selective effect of said compound on a cell type in vivo; and

formulating the compound in a medication to be administered to a patient or tested in a patient for its capacity to treat a disease affecting a tissue represented by the cell type.

The present invention permits to save on clinical trials by screening properly the compounds which would have a lesser probability of providing undesirable, even severe side effects. Further, in accordance with the present invention is provided a repertory of recombinant constructs for transforming a plurality of cell types representative of a plurality of biological tissues which comprises a reporter gene and, operably linked thereto, a promoteur comprising a minimal promoter and, upstream to said minimal promoter, a transcription factor-responsive element (TFRE) which can be bound by a transcription factor of a host cell, the diversity of the repertory being due to a plurality of TFREs.

It is further an object of this invention to provide a library of recombinant cells transformed with the constructs of the repertory.

### DESCRIPTION OF THE SPECIFIC EMBODIMENTS OF THE INVENTION

Other objects, advantages and features of the present invention will become more apparent upon reading of the following non restrictive description of preferred embodiments thereof, given by way of example only with reference to the accompanying drawings.

More specifically the present invention relates to

### BRIEF DESCRIPTION OF THE DRAWINGS

In the appended drawings:

FIG. 1 illustrates the principle of the Cell-TRAP library described in this patent application. A common reporter gene, the activity of which can be quantified, is built in a construct so as to be under the expression control of a promoter responding to particular transcription factors. (A) The library can be used to assay factor specificity after treatment with a compound. Reporters are made to respond to different factors (a to f in this example) and are used to stably transfect cells from the same line in parallel, thus generating multiple transgenic cell lines ( six in this example, one for each factors a to f) responding to different factors but in a common cellular background. (B) The library can be used to validate the effect of a compound on a particular transcription factor's activity in different cellular backgrounds. The same reporter construct is used to stably transfect different cell lines, which can for example represent different tissues or different pathological states. Each of them can then be assayed to evaluate the factor's activity in its unique background. Another obvious application not shown here would be to use the construction responding to one factor, transfect it in one cell line, and test the resulting stably transfected cell line against a multiplicity of different compounds.

FIG. 2 illustrates the expression of a reporter gene requires the activation of the transcription factor to which its promoter was made responsive. A construct containing the gene coding for GFP was built with a minimal promoter containing repeated elements allowing the binding of the estrogen receptor. GFP expression, which translates as a green fluorescence occurred only in the cell line known to contain ER (the MCF-7 line) and only in the presence of estradiol, an ER activation ligand.

FIG. 3 illustrates how the library can be built using retroviruses. (A): A plasmid is first built with a selection marker gene (open block), a reporter gene (dotted block) and a minimal promoter under the control of a particular transcription factor (filled block). it is transiently transfected into an appropriate packaging cell. (B): Virions are produced by the packaging cell which provides the missing components for producing infectious but replication-deficient retroviral particles using the plasmid as a template. The retroviral particles are then recovered in the cell culture supernatant. Highlighted is the structure of the packaged retroviral genomic RNA. Note that in a preferred embodiment of the technique, the 3′LTR carries a deletion of its promoter sequences so as not to cause spurious enhancing activities after the retroviral genome has been reverse transcribed and integrated in the final target cell's own genome. (C): The retroviral particles recovered in (B) are used to infect and transform different target cells. The retroviral genome (composed of RNA) is reverse transcribed into DNA and integrated into the host's genome. (D) The infected cell lines are submitted to selective pressure to remove untransformed cells from the total population.

FIG. 4 shows a parallel evaluation of the activity of many transcription factors using the Cell-TRAP library. The cell line MCF-7, derived from a breast cancer tumor, was stably transformed with a construct expressing GFP under the control of promoters responding to the transcription factors PPAR, p53, NFKB, NFAT and ERE, respectively. A positive control was also made, expressing GFP under the control of the strong CMV promoter. In the absence of other stimuli, it is seen that p53 has a low basal activity in this cellular context while ERE has a strong one (probably due to the presence of estrogens in the culture media). The factors PPAR, NFKB and NFAT seem silent in these conditions.

FIG. 5 shows an example of induction. Different cell lines (CEM and MCF-7 in this example) belonging to the Cell-TRAP library and containing constructs responding to the activity of the transcription factors NFKB, NFAT and ER respectively, were treated with compounds known to activate these factors. The induction in each case translates into the appearance of a green fluorescence.

FIG. 6 is a graphical demonstration of the type of global information the library can provide. The library is used to assay the effects of a compound on the pathways activating different transcription factors in different cellular contexts to validate the compound's specificity. The library allows the challenge of several pathways simultaneously in several different cellular contexts. In this figure, gray squares indicate the activation of a pathway by a compound; the degree of specificity or selectivity of the compound for a factor decreases as its screening shows that there are more and more positives along the Y axis while the tissular specificity or selectivity of the compound decreases as there are more and more positives along the X axis.

The present method is designed to evaluate the effect of any soluble molecule exerting a specific effect on the regulation of gene expression by particular transcription factors. Each cell line used in the assay is genetically modified in order to allow the quantitative evaluation of the level of activation of a specific transcriptional pathway when the cell responds to the compound. The originality of this method lies on the concomitant analysis of a compound action on different DNA regulatory elements and in different cellular contexts, while preserving the transcriptive pathways in its natural intracellular form, to any possible extent.

The final step in a transcription regulation pathway is the interaction of a gene promoter with an activated initiation complex inducing the gene transcription by a RNA polymerase. What occurs before this final step can vary depending on the nature of the signal to which the gene promoter responds: it may be that a membrane receptor was activated at the level of the cell surface, triggering a cascade of signalling events that ultimately led to an effect on a transcription factor capable of initiating the formation of an initiation complex. It could also be that such a signalling cascade reached a pre-formed complex and gave it the final signal to begin gene transcription. It could as well be that a small soluble molecule made it passed the barriers of the plasma and nuclear membranes and managed to contact an inactive transcription factor to induce in it a conformational change conferring to it the ability to induce transcription by an RNA polymerase. It could also very well be that one of these scenarios occurred, caused the synthesis of a novel transcription factor, which in turn triggered the expression of a gene of interest. All of these scenarios, as far as the ability for a given substance to act on transcription genes, are covered by the technology described in this patent: the final result which is being measured, is the expression of a reporter gene under the control of a specific transcription factor-binding DNA sequence.

At the core of the library designed for this project stands the common architecture of a reporter gene under the control of a minimal promoter containing repeated elements recognized and bound by specific transcription factors (elements like the sequences GGTTCANNNGGTTCA, recognized by a dimer of the transcription factors RXR and VDR; AGGGCANAGGTCA, recognized by a dimer of the factors PPAR and RXR, or AGGTCANNNTGACCT, recognized by a homodimer of the factor ER) (Stunnenberg, 1993). The minimal promoter, being composed of little more than a TATA box, would not by itself induce a high level of transcription of the reporter gene. When being made receptive to the induction activity of a transcription factor by having the latter's DNA-binding site being added to it, it becomes a promoter capable of driving gene expression -but only if the transcription factor in question is both present and made to provide an activation signal. If the factor binding site is present but the factor in question does not receive an appropriate activation signal, it is possible that a weak expression of the reporter gene may ensue; that weak signal, however, would be made much more obvious following an induction signal to the transcription factor. Conversely, if the factor receives not an activation signal but a repression one, the weak signal caused by the promoter on its own would likely diminish, providing a useful tool for the evaluation of a drug as a transcription antagonist.

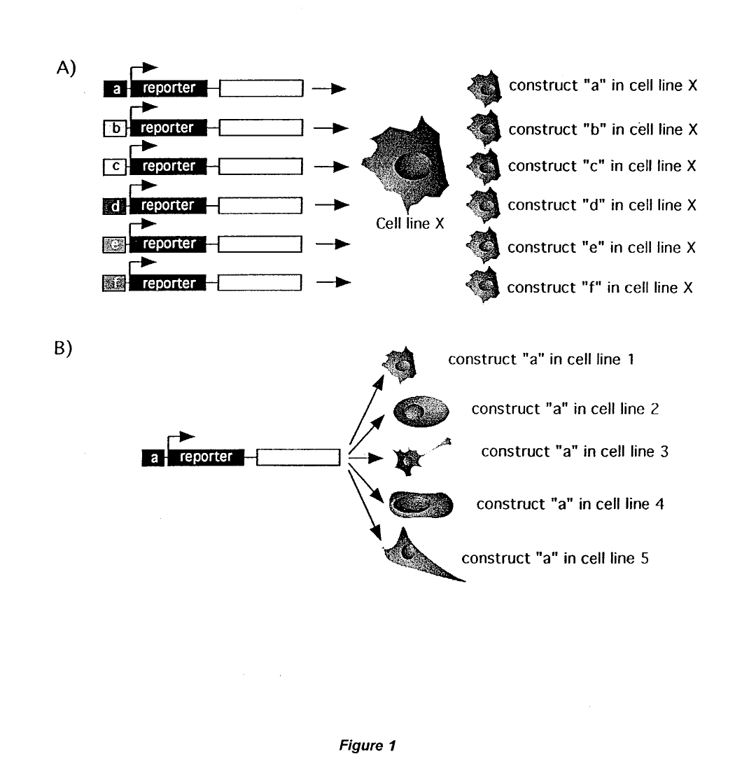

The combination of transgenic cell lines constitute the cell-based library. In the patent number U.S. Pat. No. 5,863,733, Foulkes et al. claims to have invented “a method of determining whether a chemical not previously known to be a modulator of protein biosynthesis specifically transcriptionally modulates the expression of a gene-of-interest ( . . . )”. The present inventors do not agree that the method reported in this patent is specific. In order to do so, one has to evaluate (1) the other transcriptional pathways potentially submitted to the influence of the compound of interest (2) the transcriptional activation of the pathway in other cell types. The present strategy is carefully taking these points into consideration by using a multi-cellular assay in which library of transgenic cells is simultaneously monitored instead of using one construct at the time. In this system, several pathways were monitored as well as one peculiar pathway in several tissue origins, as schematized in FIG. 1a) and b). It is believed that multiparameter analysis is much more likely to give a set of data capable to addressing the problem of specificity or selectivity of action. The multiparameter analysis tool presented in the present patent is also a very good tool to evaluate the potential transcriptional “side effects” of a compound.

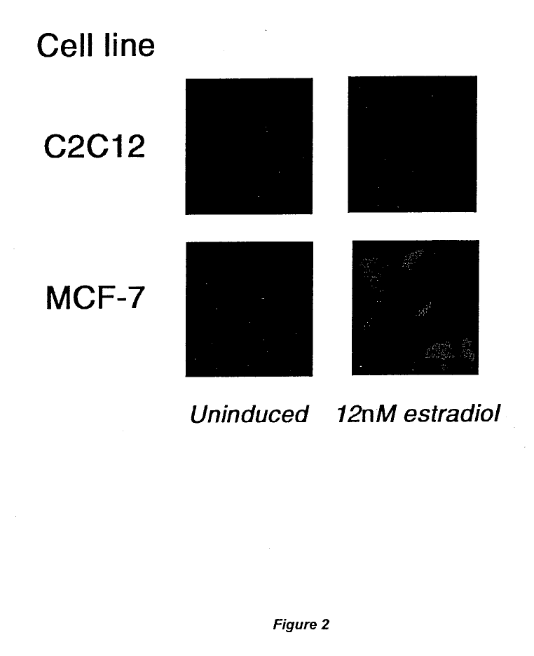

FIG. 2 shows how the expression of a reporter gene (in this case Green Fluorescent Protein (GFP)) can be made dependent of a particular transcription factor (in this case the estrogen receptor ER) in a ligand-specific way. Two cell lines are shown in this example: C2C12 is a myoblast-derived cell line and is not known for its great ER activity; the MCF-7 cell line, on the other hand, is derived from a breast carcinoma and harbors ER activity. Both cell lines were stably transfected with a construct in which GFP expression is under the control of a minimal promoter containing repeated elements to which ER can bind. The construct also contains a selection marker which confers resistance to the antibiotic geneticin (G-418, Roche Molecular Biochemicals) so that transfected cells could be separated from non-transformed ones. As would be expected, no signal (a green fluorescence) can be seen in either cell line when no ligand is used (ER will induce gene transcription only when it is activated by an estrogen such as estradiol). When estradiol is added, only the cell line containing endogenous ER (the MCF-7 line) can respond to the ligand and induce gene expression. This demonstrates that it can be shown that a compound can induce the activity of a transcription factor and that this will occur in particular cell lines.

To allow the study of the effect of a compound on the activity of many transcription factors, we had to design a variety of reporter constructs, each of which making the GFP reporter gene responsive to a different transcription factor or family thereof. A common framework was adopted, in which a GFP gene was built immediately downstream of a minimal promoter. Downstream of the GFP, a selection marker was introduced to confer resistance to the antibiotic Geneticin. A site shortly upstream of the TATA box of the minimal promoter was reserved for the introduction of repeated DNA-binding sites for the different transcription factors to be studied. These latter sites were first synthesized as double-stranded oligonucleotides carrying sequences known to be specifically recognized by certain transcription factors or families thereof (See appendix 1). They were then di- or multimerized, when desirable, by ligation and sub-cloned into a shuttle vector, in which they were sequenced to ascertain their actual sequence. The number of copies (normally from 2 to 10 copies) depends on the factor studied. Different types of multimers were produced and the best ones were selected; in most cases, a single repeat was sufficient (e.g. the p53 construct seen in the different figures has only one repeat). The monomeric or multimerized DNA-binding sites were freed of the vector and introduced in front of the TATA box of the minimal promoter of the reporter construct. This provided a collection of reporter genes, each under the transcriptional control of a different transcription factor or family thereof.

Each one of these constructs was then used to generate multiple transgenic or recombinant cell lines, so that the factor represented by each construction could be studied in different cellular contexts. This was achieved in two ways (although more could be used and still reflect the same idea of generating stably-transformed libraries): stable transfection followed by selection with the antibiotic was the first; integration using a retroviral vector was the second. For the latter operation, the reporter construct was assembled in a murine retroviral backbone (MOMLV) modified in certain ways. First, the retroviral backbone has been made inapt to replication by deletion of the genes gag, env and pol. Second, its 3′ long terminal repeat (or LTR) region has been mutated so that its natural strong promoter activity is missing. The latter maneuver is made necessary because of the risk that upon integration, an unhindered 3′LTR (whose sequence would end up upstream of the inserted reporter construct) could drive the expression of the reporter gene independently of the minimal promoter or of the transcription factor under study. Once the different reporter constructs have been introduced in such a retroviral backbone, they can be transfected in a packaging cell line such as the Fred Hutchison Cancer Research Center's line PT67 (Miller, 1998). This line has been modified to carry the genes missing for a proper retroviral replication; furthermore it carries surface markers which confers to any virus being produced in it a very wide spectrum of infectivity tissue-wise. To help matters further, it is possible to co-transfect the packaging cell line with an expression vector for the vesicular stomatitis virus protein G, which helps make the virus even more readily integrated by target cells because of a membrane fusion mechanism rather than one proceeding through membrane receptors. Viral particles will bud from the packaging cell line, each capable of infecting a wide variety of cell lines and tissues, in the genome of which the appropriate reporter constructs will stably integrate. (Once integrated, the retroviral genomes will no longer produce retroviruses, of course).

The transgenes, be they transferred to the target cells by transfection, retroviral infection, or any other technique, will integrate in different locations in the host's genome. This could lead to epigenetic modulation effects on gene expression. Such a problem can be circumvented in two ways. First, insulators could be used to flank the reporter constructs, thus keeping the latter safe from interference by their chromosomal surroundings (Pikaart, 1998; Udvardy, 1999). Second, the random integration of the transgene and subsequent selection for antibiotic resistance will generate a polyclonal population in which integration in unfavorable sites (where silencing can occur) should be compensated by integration in stimulating sites (close to strong enhancer elements). Furthermore, should the general background level of activation be too weak, it is always possible to enrich the population in more active cells by FACS separation.

It is noted that the activity of a transcription factor in the activity of a putative therapeutic compound can be confirmed by interfering with its binding to the responsive promoter element in a cell under study. The same constructs could then be made for this purpose, replacing the functional responsive-elements with mutated ones that is no longer bound by its assigned factor (See appendix I, “mut” oligos).

Detailed Protocols

Retroviral Expression Vector Construction Strategy

Construction of the vector was performed according to standard practices in the field. A retroviral vector previously described in the literature (Hooijberg et al., 2000) was obtained under licence from its developers and used as starting material for this purpose. First, the GFP reporter gene from this retrovector was changed to a different version of GFP (coding for a protein whose excitation wavelength is at around 400 nm rather than 490 nm). The promoter driving the expression of the protein was also changed to a deleted version of the CMV promoter retaining little beyond the TATA box. Upstream of this promoter, a unique Nru I site was inserted to allow the introduction of DNA-binding sites for different transcription factors. A cassette coding for G-418 resistance, under the control of the PGK promoter, was added 3′ of the GFP gene in order to serve as a selection marker in transfected cells.

DNA-binding sites such as those listed in appendix 1 were first synthesized as complementary oligonucleotides and then annealed. Each oligonucleotide synthesized carried the sequence of two DNA-binding sites for the same factor arranged in tandem. The annealed oligonucleotides were then ligated in concatemers and cloned in a shuttle vector. The resulting plasmids were transformed in E. coli and cultured separately, and a few clones for each construct had its DNA purified and sequenced. The appropriate tandem arrays of each transcription factor DNA-binding site were then cut out of the shuttle vector using restriction enzymes, and introduced into the unique Nru I site of the retroviral vector.

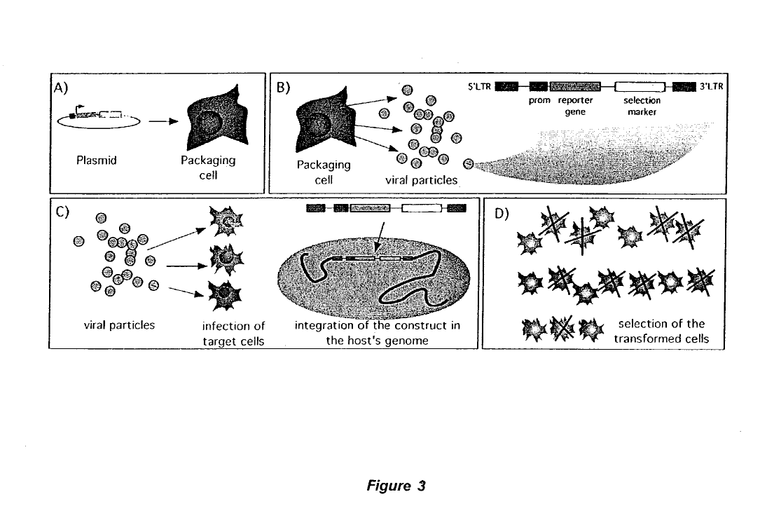

The final retroviral constructions are represented in FIG. 3.

Stable Transfection

A cell library such as the one described in the present patent application can be produced by stable transfection. This was demonstrated by recovering from the above retroviral construction the relevant DNA fragment (from a position downstream of, and excluding, the viral 5′LTR and packaging signal, and down to, and including, the mutated 3′LTR). Such as DNA fragment was then introduced into final target cells by a variety of means known to those knowledgeable in the art, and adapted to each cell type. These means included, for example, electroporation, calcium phosphate co-precipitation, and use of commercial transfection reagents such as Qiagen's superfect (Qiagen Inc.). The transfected cells were then submitted to a selective pressure by treatment with increasing concentrations of geneticin (Roche biochemicals). Since only stably transfected cells expressing the resistance gene would survive for many generations to the treatment, there was a polyclonal amplification of the transformed cells to the detriment of the untransformed ones, which died in the process. Each stably transfected cell line, after polyclonal expansion, was kept as a frozen stock for later use.

Retroviral Stock Production

Retroviral vectors were transiently transfected into packaging cell line PT67 (Miller, 1998) according to protocols well-known in the field. A successful transfection was made apparent after 48 hours, as a very large part of the transfected PT67 cells expressed GFP and could be seen to generate an intense green fluorescence under the excitating light of a laser producing light at a wavelength of 405 nm. The cells were allowed to produce viral particles for a few days as the culture's virus-rich supernatant is recovered after 48, 72 and 96 hours. The viral stocks can be concentrated by a variety of methods, such as centrifugation on commercially available concentration columns or ultracentrifugation at 50 000 g for two hours. The viral stocks can be frozen at any time.

Mammalian Cell Infection

Viral particles produced from the PT67 the packaging cell line can infect a very wide spectrum of mammalian cells (see FIGS. 3a) to c)). The recovered supernatant, fresh or from a thawed frozen stock, is added to the culture medium of the final target cells and allowed to infect them. Infection is monitored using fluorescent microscopy which detects the expression of GFP. Antibiotic selection then allows the elimination of untransformed cells (FIG. 3d).

Use of Cell Library and Activation of Specific Transcriptional Pathways

Lead optimisation is one of the major area in drug development. Our method can be used to compare the specific pathways induced by a parental compound and its derivatives. Synthetic derivatives of a compound can exert more potent action than the parental product. In a similar manner, these derivatives can also induce undesired secondary effects. The cell library can be used to delineate the more potent and more specific compounds among a list of structurally related products and help to focus the next round of compound synthesis.

The utilization of a particular vector for transformation of several cell lines of different origin will allow to investigate the effect of a compound upon activation of a specific transcription factor family in different tissue origin context and permit the profiling of response among different cell lineage. Tissue specificity of action can be a strong guideline for hierachization of several structurally related compounds inducing variable set of responses.

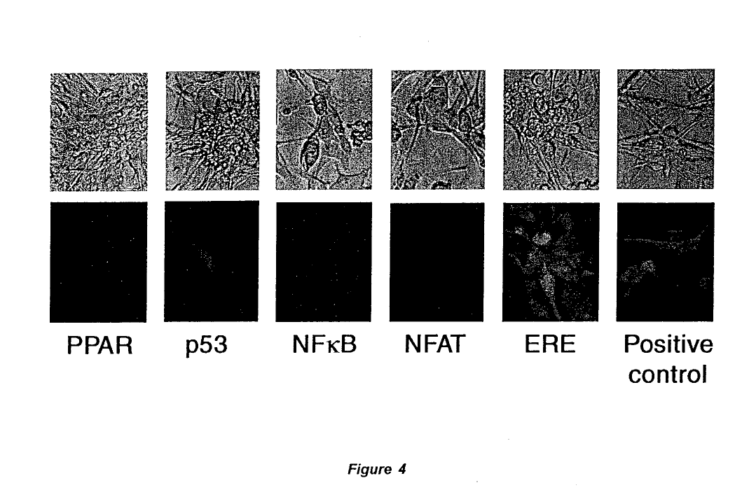

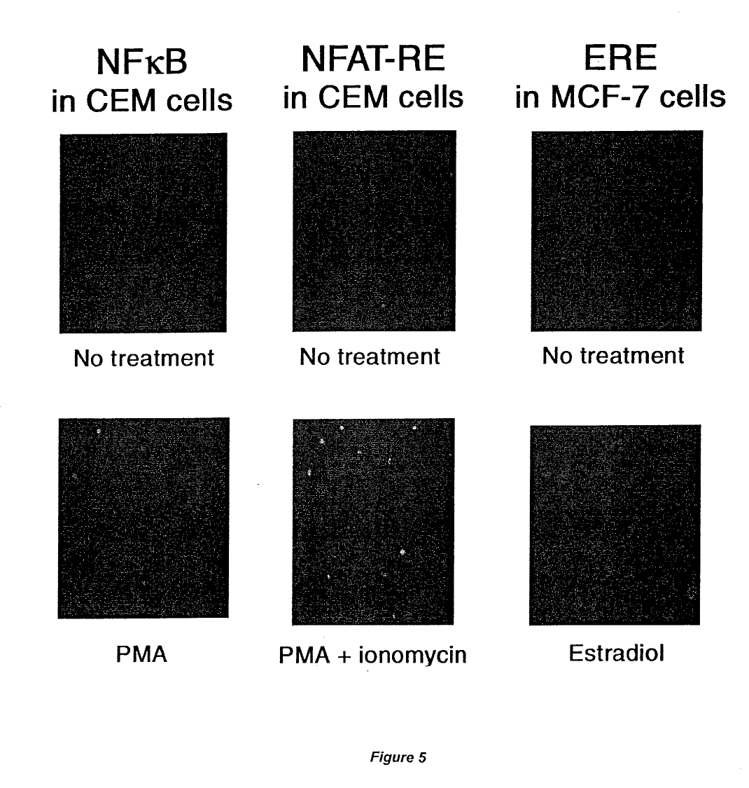

The transcriptional activation of a gene constitute the end of a biological cascade originating from extracellular activators such as cytokines, steroids hormones, peptidic hormones, prostaglandins, chemicals, which upon interacting with a cellular component modify the intracellular phosphorylation state, leading to genomic expression changes in the nucleus. FIG. 4 shows the differential expression of transcription factors as evaluated by the present invention, taking as an example MCF-7 cells grown in the presence of estradiol. The factor p53 show a low basal activity while ERE factor has a strong activity under these conditions. As shown in FIG. 5, the activation of NFkB or NFAT cascade is different from activation of estradiol receptor cascade. Thus, the fact that a compound leading accumulation of GFP in a cell line transformed with NFkB/GFP or NFAT/GFP reporter gene while doing nothing in ER/GFP cell lines would indicate that potential targets have been hit along the NFkB or NFAT transcriptional cascade. By comparison of activation profiles along the cell library, one could determine the potential targets involved in reporter gene activation by the compound and orient further research for elucidation of the mechanism of action of the compound of interest and its effector targets.

Cell library can be used to screen compounds in a wide variety of platforms. Biological processes occurring in living cells have been demonstrated in microplate, nanoplate, microchip, membrane and gel matrix environments. Accordingly, our cell library could take advantage of every cultivating system that allow a mid to large scale screening process to take place.

The pharmaceutical industry have generated thousands of compounds in the search of therapeutic leading agents in order to treat and cure diseases. However the process of screening the compound candidates to detect potential agent is difficult, takes a considerable amount of time and requires major capital investments. Reducing the time for drug discovery is becoming a paramount for the pharmaceutical industry. A specific gene regulatory agent constitutes a major lead for therapeutic purposes. Screening compounds on the basis of its specificity of transcriptional activity profile confer to the present procedure the capacity of preventing million dollars expenses on possible wrong compound and save a tremendous amount of time and a money for the industry.

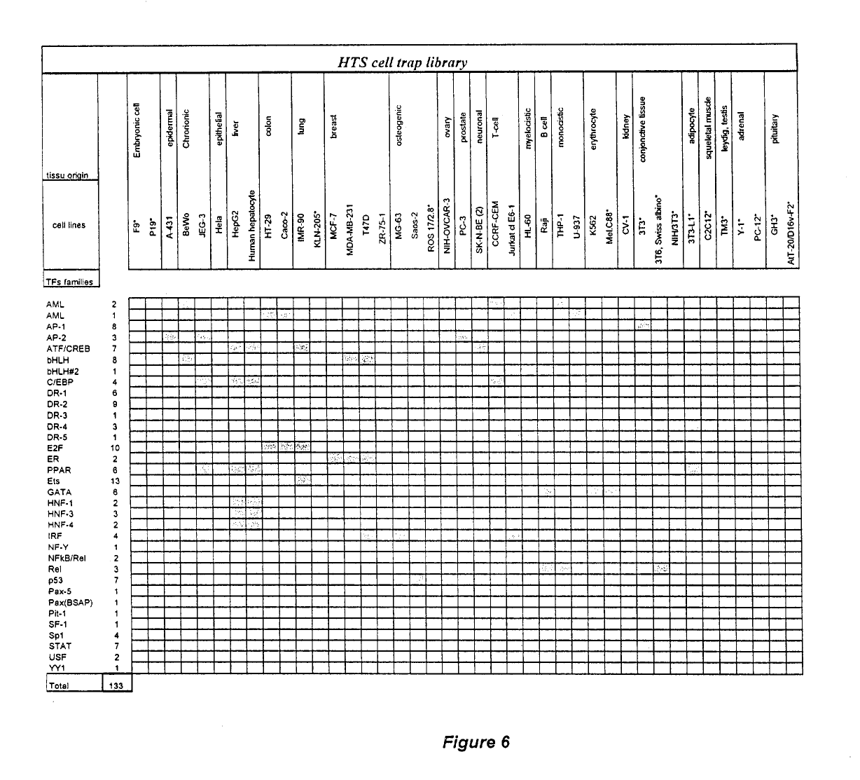

FIG. 6 shows that it is possible to obtain an activation profile for a given compound for a given transcription facotr and for a given cell. The lesser the number of filled boxes in the X and Y axis, the more selective is a compound for a given transcription factor and a given target cell type, respectively.

Let's imagine a real situation case: estrogens, for example, are intended to be used for inhibiting restenosis (which involves vascular smooth cell proliferation) occurring upon angioplasty. One wants to select the proper estrogen(s) for such therapeutic effect. Cell lines representative of the in vivo target tissues or cells (e.g. of common tissue origin) are included in the screening assay. For instance, a primary smooth muscle cell line would be transformed with constructs that would comprise at least a ER construct. A plurality of estrogen-like compounds are used to screen the library which would comprise also other cell types susceptible to respond to estrogens (breast cell lines, for example). Since estrogens activate at least two types of receptors (estrogen receptors α and β) and since these receptors may be coupled to different transcription pathways, one may see a different transcription profile for estrogens, providing the user with a selection of one or more estrogens that are preferred over others because of their selectivity for the type of receptors that is estrogen-responsive in vascular smooth muscle cells. If the candidate compound shows the best potential of activity but lacks selectivity, the information on the profile of the compound may indicate or suggest which route of administration should be favored (ex.: an in situ treatment).

In the above example, the compounds are known, but it is contemplated that unknown compounds can be screened to obtain a selectivity profile leading to the judicious choice and study of a therapeutic compound.

Although the present invention has been described hereinabove by way of preferred embodiments thereof, it can be modified, without departing from the spirit and nature of the subject invention as defined in the appended claims.

[image]

[image]

[image]

[image]

[image]

[image]

[image]

[image]

[image]

What is claimed is:

1. A method for selecting and producing a therapeutic compound which is presumed selective for one or a restricted set of given transcriptional pathways and of given cell types or for validating a putative useful compound as a selective therapeutic compound towards said one or restricted set of given transcriptional pathways and cell types, which comprises:
providing a construct which comprises a reporter gene, the expression of which is driven in a host cell by a promoter capable of directing transcription of the gene operably linked thereto upon activation, which promoter comprises a minimal promoter, and upstream to said minimal promoter, a transcription factor-responsive element which is capable of affecting the activity of the minimal promoter upon binding by a transcription factor endogenously produced, activated or inactivated by the host cell upon contacting by the compound; the construct being provided for a plurality of transcription-factor responsive elements for a given cell line and for a plurality of cell lines representative of different tissues;
inserting each construct into the genome of the host cell of each cell line, thereby obtaining a library of recombinant cell lines;
contacting the compound with the library of recombinant cell lines;
detecting a change in the expression of the reporter gene occurring in one recombinant cell line or in a subset of recombinant cell lines and not in other cell lines of the library as an indication of a selective effect of said compound on a cell type in vivo; and
formulating the compound in a medication to be administered to a patient or tested in a patient for confirmation of its capacity to selectively treat a disease affecting a tissue represented by the cell type.

2. The method of claim 1, wherein the construct is provided for at least two transcription factors selected from appendix I.

3. The method of claim 2, wherein the construct is provided for at least two transcription factor-responsive elements selected from appendix I.

4. The method of claim 1, 2 or 3, wherein the transcription factor-responsive element is present in more than one copy placed in tandem.

5. The method of any one of claims 1 to 4, wherein the construct is a retroviral construct modified to be incapable of transmitting a retroviral disease and to be incapable of acting as a promoter for the reporter gene.

6. The method of any one of claims 1 to 5, wherein detecting the change in the expression of the reporter gene is made through the detection of the formation of an amplified product by a polymerase chain reaction primed with amplification primers complementary to an expressed sequence comprising the reporter gene, or is made through the detection of the gene product of the reporter gene.

7. The method of claim 6, wherein the detection of the gene product of the reporter gene is made through the detection of the formation of an immune complex with an antibody directed against the gene product.

8. The method of claim 6, wherein the detection of the gene product of the reporter gene is made through the detection of a luminescent or fluorescent gene product.

9. The method of claim 8, wherein the reporter gene is coding for the green fluorescent protein (GFP).

10. A repertory of recombinant constructs for transforming a plurality of host cell types representative of a plurality of biological tissues which comprises a reporter gene and, operably linked thereto, a promoter comprising a minimal promoter and, upstream to said minimal promoter, a transcription factor-responsive element (TFRE) which can be bound by a transcription factor of a host cell, the diversity of the repertory being due to a plurality of TFREs.

11. The repertory of claim 10 wherein the TFRE comprises any one of the TFREs listed in appendix I.

12. The repertory of claim 10 or 11, wherein the reporter gene is a gene encoding green fluorescent protein.

13. The repertory of any one of claims 10 to 12, wherein the construct is a retroviral construct.

14. A library of recombinant cells transformed with the constructs of the repertory of any one of claims 10 to 13.
